(19) United States
(12) Patent Application Publication
(10) Pub. No.: US 2006/0281686 A1
(43) Pub. Date: Dec. 14, 2006

(54) Aza-peptides

(76) Inventors: John Jairo Lopez Areiza, Lausanne (CH); Thomas Rueckle, Plan-les-Ouates (CH); Claudio Soto-Jara, Friendswood, TX (US)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(21) Appl. No.: 10/537,246

(22) PCT Filed: Dec. 1, 2003
(86) PCT No.: PCT/EP03/15033

§ 371 Date: Apr. 7, 2006

(30) Foreign Application Priority Data
Dec. 2, 2002 (EP) 02102673.7

(51) Int. Cl. A61K 38/08; C07K 7/06

(52) U.S. Cl. 514/17; 530/330

(57) ABSTRACT

Provided are peptides comprising at least one azaamino acid and having β-sheet breaking ability, useful in the treatment and prevention of diseases such as alzheimer's disease, Dementia Pugilistica (including head trauma), Hereditary Cerebral Haemorrhage with amyloidosis of the Dutch type (HCHWA-D) and Vascular Dementia with amyloid angiopathy.

### FIELD OF INVENTION

The invention relates to the field of β-sheet breaking peptides, particularly their use in the treatment of diseases such as Alzheimer's disease, Dementia pugilistica (including head trauma), Hereditary Cerebral Haemorrhage with amyloidosis of the Dutch type (HCHWA-D) and vascular dementia with amyloid angiopathy.

### BACKGROUND OF THE INVENTION

Alzheimer's disease (AD), first described by the Bavarian psychiatrist Alois Alzheimer in 1907, is a progressive neurological disorder that begins with short-term memory loss and is characterized by a progressive decline in cognitive function and behaviour. Progression of the disease leads to disorientation, impairment of judgment, reasoning, attention and speech and, ultimately, dementia. The course of the disease usually leads to death in a severely debilitated, immobile state between four and 12 years after onset. AD has been estimated to afflict 5 to 11 percent of the population over age 65 and as much as 47 percent of the population over age 85. The societal cost for managing AD is very high, primarily due to the extensive custodial care required for AD patients. Despite continuous efforts aimed at understanding the physiopathology of AD, there is currently no treatment that significantly retards the progression of the disease.

Pathologically, AD is characterized by the presence of distinctive lesions in the victim's brain, revealed on autopsy. These brain lesions include abnormal intracellular filaments called neurofibrillary tangles (NTFs) and extracellular deposits of amyloidogenic proteins in senile, or amyloid, plaques. Amyloid deposits are also present in the walls of cerebral blood vessels of AD patients. The major protein constituent of amyloid plaques has been identified as a 4.3 kiloDalton peptide called β-amyloid peptide (Aβ)¹. Diffuse deposits of Aβ are frequently observed in normal adult brains, whereas AD brain tissue is characterized by more compacted, dense-core β-amyloid plaques.² These observations suggest that Aβ deposition precedes, and contributes to, the destruction of neurons that occurs in AD³. In further support of a direct pathogenic role for Aβ, β-amyloid has been shown to be toxic to mature neurons, both in culture and in vivo⁴.
¹Selkoe, Science, 275, 1997, 630-631

²Masters, C. et al. Proc. Natl. Aced. Sci. USA, 82, 1985, 4245-4249

³Davies, L et al. Neurology, 38, 1988, 1688-1693

⁴Kowall, N. W. et el. Proc. Natl. Aced. Sci. USA, 88, 1991, 7247-7251

Patients with hereditary cerebral haemorrhage with amyloidosis-Dutch-type (HCHWA-D), which is characterized by diffuse β-amyloid deposits within the cerebral cortex and cerebrovasculature, have been shown to have a point mutation that leads to an amino acid substitution within Aβ.⁵
⁵Levy, E. et al. Science, 248, 1990, 1124-1126 82-1888.

Aβ has also been implicated in vascular dementia with amyloid angiopathy⁶ and dementia pugilistica.⁷
⁶Maury, C. P.; Lab Invest., 72, 1995, 4-16

⁷Jordan, B. D. Semin. Neurol., 20, 2000, 179-85

Natural Aβ is derived by proteolysis from a much larger protein called the amyloid precursor protein (APP)⁸. The APP gene maps to chromosome 21, thereby providing an explanation for the β-amyloid deposition seen at an early age in individuals with Down's syndrome, which is caused by trisomy of chromosome 21⁹.
⁸Kang J. et al. Nature, 325, 1987, 733

⁹Mann, D. M: Histopeathology, 13, 1988, 125-37

Naturally-occurring Aβ, derived from proteolysis of APP, is 39 to 43 amino acid residues in length, depending on the carboxyl-terminal end point, which exhibits heterogeneity. The predominant circulating form of Aβ in the blood and cerebrospinal fluid of both AD patients and normal adults is Aβ1-40¹⁰. However, Aβ1-42 and aβ1-43 are also found in β-amyloid plaques¹¹.
¹⁰Shoji, M. et al. Science, 258, 1992, 126

¹¹Mori, H. et al. J. Biol. Chem., 267, 1992, 17082

Considerable evidence has accumulated that the pathogenicity of Aβ results from a change in protein conformation¹². It is believed that a critical event leading to pathology in Alzheimer's disease, Vascular Dementia with amyloid angiopathy and HCHWA-D is the refolding of a natural and non-pathogenic protein, to yield a pathogenic form. The refolding alters the secondary and tertiary structure of the protein without changing its primary structure.
¹²Soto J. Mol. Med., 77, 1999, 412-418

Amyloid is a generic term that is applied to fibrillar aggregates that have a common structural motif: a β-pleated sheet conformation¹³. These aggregates exhibit special tinctorial properties, including the ability to emit a green birefringent glow after staining with Congo red, and the capacity to bind the fluorochrome thiofiavin S¹⁴. These tinctorial properties form the basis of assays used to detect β-amyloid deposits.
¹³Serpell et al. Cell Mol. Life Sci., 53, 1997, 871-887

¹⁴Soto, C. et al. J. Biol. Chem., 270, 1995, 3063-3067.

One approach to the treatment and prevention of Alzheimer's disease has been to develop short peptides having some sequence homology to the natural protein sequence believed to be involved in amyloid formation, but also having one or more amino acids that disfavour or destabilise the formation of β-pleated sheet conformations¹⁵. The peptides prevent the aggregation of β-amyloid, and thereby prevent its cytotoxic effects. This approach has been suggested in Alzheimer's disease and in prion-related disorders^{16,17,18} and has lead to the β-sheet breaking peptides¹⁹ shown below, amongst others: 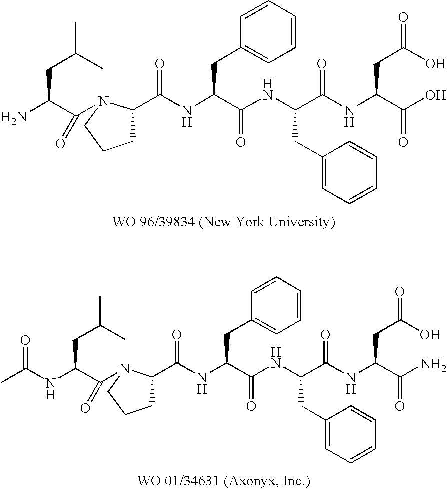
¹⁵Wisniewski et al. Biochemical Society Transactions, 30 (4), 2002, 574-578

¹⁶WO 96/39834 (New York University)

¹⁷WO 01/34631 (Axonyx Inc.)

¹⁸U.S. Pat. No. 5,948,763 (New York University)

¹⁹Hentényl et al. Journal of Molecular Structure, 542, 2001, 25-31

U.S. Pat. No. 6,319,498 (Praecis Pharmaceuticals) proposes β-sheet breaking peptides based on Aβ, and exemplifies amino-terminally-biotinylated peptides. U.S. Pat. No. 6,303,567 (Praecis Pharmaceuticals) proposes peptides based on the β-amyloid peptide, but consisting entirely of D-amino acids, as β-sheet breaking peptides.

While the known β-sheet breaking peptides have provided valuable information and may have utility in treating Alzheimer's disease, the development of peptide drugs is severely limited by the fact that natural peptides are subject to degradation and rapid metabolism in the gut, the liver and in circulation. Furthermore, the desired site of action for treatment of many amyloid-related disorders is in the brain, and peptides, like many other molecules, may have difficulty penetrating the blood brain barrier. The brain itself is also replete with peptidases, which degrade peptide molecules.

### SUMMARY OF THE INVENTION

It is an object of the invention to provide a β-sheet breaking peptide with improved pharmacological profile.

In a first aspect, the invention provides a compound of the general Formula I: 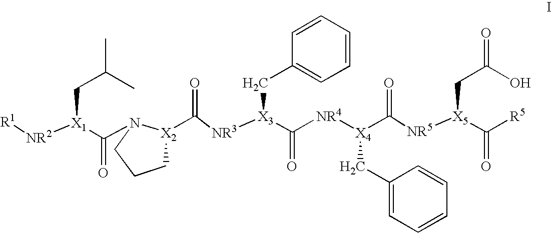
wherein:

R¹ is selected from H, optionally substituted C₂-C₆ acyl and optionally substituted C₁-C₆ alkyl;

R², R³, R⁴ and R⁵ are independently selected from H and optionally substituted C₁-C₆ alkyl;

R⁶ is selected from OH and NR⁷R⁸, wherein R⁷ and R⁸ are independently selected from H or optionally substituted C₁-C₆ alkyl;

X₁, X₂, X₃, X₄ and X₅ are independently selected from CR⁹ or N wherein R⁹ is selected from H and optionally substituted C₁-C₆ alkyl and with the condition that at least one among X₁, X₂, X₃, X₄ and X₅ is N;

In a second aspect, the invention provides a compound of the general Formula I for use as a medicament;

In a third aspect, the invention provides a pharmaceutical composition comprising a compound of Formula I, together with a pharmaceutically acceptable excipient or carrier.

In a fourth aspect, the invention provides a use of a compound of Formula I for the preparation of a medicament for the treatment or prevention of a disease or condition selected from Alzheimer's disease, Dementia Pugilistica (including head trauma), Hereditary Cerebral Haemorrhage with amyloidosis of the Dutch type (HCHWA-D) and vascular dementia with amyloid angiopathy.

In a fifth aspect, the invention provides a use of a compound of Formula I for the treatment or prevention of a disease or condition selected from Alzheimer's disease, Dementia Pugilistica (including head trauma), Hereditary Cerebral Haemorrhage with amyloidosis of the Dutch type (HCHWA-D) and Vascular Dementia with amyloid angiopathy.

In a sixth aspect, the invention provides a method of treating Alzheimer's disease, Dementia Pugilistica (including head trauma), Hereditary Cerebral Haemorrhage with amyloidosis of the Dutch type (HCHWA-D) and Vascular Dementia with amyloid angiopathy, comprising administering an effective amount of a compound of Formula I to a patient in need thereof.

In a seventh aspect, the invention provides a use of a compound of Formula I for the preparation of a medicament for the treatment of a disease associated with abnormal protein folding into amyloid and amyloid-like deposits.

In an eighth aspect, the invention provides a use of a compound of Formula I for the treatment of a disease associated with abnormal protein folding into amyloid and amyloid-like deposits.

In a ninth aspect, the invention provides a method of treating a disease associated with abnormal protein folding into amyloid and amyloid-like deposits, comprising administering an effective amount of a compound of Formula I to a patient in need thereof.

In a tenth aspect, the invention provides a method for the preparation of aza-peptides through a process comprising at least the step reacting an aza-amino acid building block of Formula (II) with an amino acid, an aza-amino acid, a peptide, an aza-peptide or an azatide to form a aza-peptoidic bond through aza-peptide coupling:

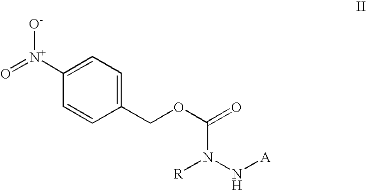

wherein:

R is selected from H and C₁-C₆ alkyl, preferably H;

A is any functional group of an amino acid, optionally protected by a protecting group;

and R and A can form an optionally substituted C₃-C₆-heterocycloalkyl ring, preferably pyrazolidinyl-, diazetidine-, hexahydropyridazine-, 1,2-diazepane-, or 1,2-diazoctane group.

In an eleventh aspect, the invention provides aza-peptide building blocks having a Formula selected from III, IV, V and VI:

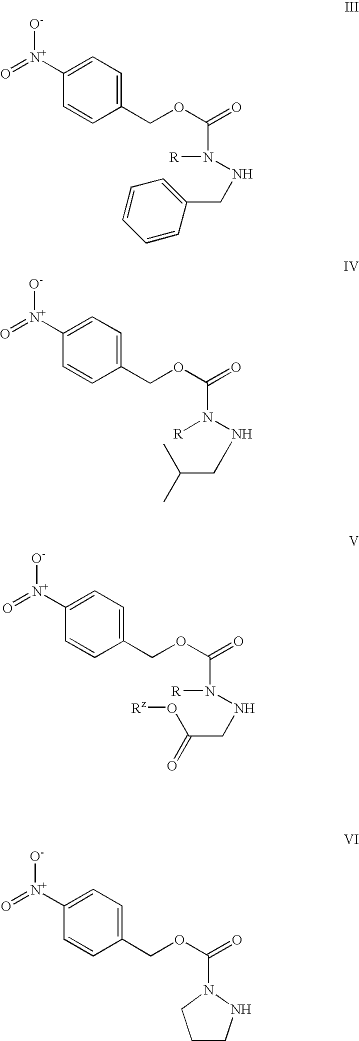

wherein:

R is selected from H and C₁-C₆ alkyl, preferably H;

R^{Z} is selected from —CH₂—CH═CH₂ and -tert-butyl.

In a twelfth aspect, the invention provides a method for the preparation of aza-peptides of Formula I.

### DETAILED DESCRIPTION OF THE INVENTION

The compounds of the invention are β-sheet breaking peptides with improved pharmacological profile over known β-sheet breaking peptides.

β-sheet breaking activity can be detected using, for example, an in vitro assay, such as that described by Soto et al.¹⁴, which measures the ability of test compounds to prevent amyloid fibril formation.
¹⁴Soto, C. et al. J. Biol. Chem., 270, 1995, 3063-3067.

Amyloid fibrils are cytotoxic, inducing cell death by apoptosis²⁰. Compounds of the invention can be tested for their ability to prevent cell death induced by amyloid fibrils. Results are reported in the Examples.
²⁰Yankner, B. A.; Neuron, 16, 1996, 921-932

A compound having an improved pharmacological profile is considered to be a compound having an increased in vitro activity, as measured by either or both of the in vitro assays described herein, an increased stability in plasma and/or in brain homogenate, or an increased ability to prevent amyloid deposition in vivo in rat brain, as compared with known compounds. “Improved” encompasses those compounds showing an increase in any one of these parameters, or in more than one. Preferably the improvement will be a statistically significant one, preferably with a probability value of <0.05. Methods of determining the statistical significance of results are well known and documents in the art, and any appropriate method may be used.

In a preferred group of compounds of Formula I, R¹ is selected from formyl, acetyl, propionoyl, butyroyl, aminoacetyl, methylaminoacetyl, dimethylaminoacetyl, aminoethyl, methylaminoethyl, dimethylaminoethyl and methyl. Particularly preferably R¹ is selected from acetyl, methylaminoacetyl, dimethylaminoacetyl, most preferably acetyl.

In another preferred group of compounds of Formula I, R⁶ is NHMe or NH₂, particularly preferably NH₂.

In another preferred group of compounds of Formula I, R², R³, R⁴ and R⁵ are selected from H, methyl and ethyl, particularly preferably H or methyl. In a particularly preferred group of compounds, R², R³, R⁴ and R⁵ are H. Another particularly preferred group of compounds, R³ is methyl. In a particularly preferred group of compounds, R³ is methyl and R², R⁴ and R⁵ are H.

In another preferred group of compounds of Formula I, X₁, X₂, X₃, X₄ and X₅ are independently selected from CR⁹ or N wherein R⁹ is preferably H or methyl and with the condition that at least one among X₁, X₂, X₃, X₄ and X₅ is N;

In another preferred group of compounds of Formula I, X₁, X₃, X₄ and X₅ are independently selected from —CH or N and X₂ is N;

In another preferred group of compounds of Formula I, X₁, X₂, X₄ and X₅ are —CH or N and X₃ is N;

In another preferred group of compounds of Formula I, X₁, X₄ and X₅ are —CH or N and X₂ and X₃ are N;

The compounds of the invention may be isolated and purified as salts. Such salts fall within the scope of the invention. For the purposes of administration to a patient, it is desirable that the salts be pharmaceutically acceptable.

The compounds of the invention can be administered as salts. Such salts include: salts of carboxyl groups or acid addition salts of amino groups of the peptide of the invention. Salts of a carboxyl group may be formed by means known in the art and include inorganic salts, for example, sodium, calcium, ammonium, ferric or zinc salts, and the like, and salts with organic bases as those formed, for example, with amines, such as tri-ethanolamine, arginine or lysine, piperidine, procaine and the like. Acid addition salts include, for example, salts with mineral acids such as, for example, hydrochloric acid or sulfuric acid, and salts with organic acids such as, for example, acetic acid or oxalic acid.

“Aza-aminoacid” is defined as a peptidomimetic moiety formed by the replacement of an α-carbon of an amino acid with a nitrogen atom. When inserted in a peptide sequence, the aza-aminoacid, leads to an “aza-peptoidic” bond at the position where the α-carbon has been replaced.

“Aza-peptide” is defined as a peptide containing one or more aza-aminoacid(s).

“Azatide” is defined as a “pure” aza-peptide, i.e. a peptide which is only constituted by aza-aminoacids.

“Normal amino acid” refers to an amino acid in which the α-carbon of an amino acid is not replaced by a nitrogen.

“Peptoid unit” refers to either an amino acid or an aza-amino acid which constitutes the aza-peptide backbone. The total number of “peptoid units” within an aza-peptide determines the aza-peptide length. For example, a diaza-peptide has a total length of two peptoid units, a penta-azapeptide has a total length of five peptoid units. By extension, for an azatide, the number of peptoid units corresponds to the total number of aza-amino acids present in the sequence.

“Aza-peptide coupling” refers to the coupling of an amino-acid or of an aza-amino acid to either an another amino-acid, an another aza-amino acid, a peptide, an aza-peptide or to an azatide through the formation of a peptidic bond or an “aza-peptoidic” bond.

“Functional group” of an amino acid refers to the side chain which is specific to each particular amino acid. For example, the functional group of Phenylalanine is benzyl and of Leucine is isobutyl.

“Chiral derivative” refers to any substitution of a normal amino acid (L-enantiomer) by the corresponding D-enantiomer.

The amino acids are represented by their corresponding one letter code, i.e. D is Aspartic acid, F is Phenylalanine, L is Leucine, P is Proline.

The superscript “a” following a one-letter code indicates an aza-amino acid linkage.

“C₁-C₆ -alkyl” refers to monovalent branched or unbranched alkyl groups having 1 to 6 carbon atoms. This term is exemplified by groups such as methyl, ethyl, n-propyl, isopropyl, n-butyl, isobutyl, tert-butyl, pentyl, hexyl and the like.

“C₁-C₅ -alkyl” refers to monovalent branched or unbranched alkyl groups having 1 to 5 carbon atoms. This term is exemplified by groups such as methyl, ethyl, n-propyl, isopropyl, n-butyl, isobutyl, tert-butyl, propyl, pentyl and the like.

“C₂-C₆ Acyl” refers to a group —C(O)R where R includes “C₁-C₅-alkyl” groups.

“C₃-C₆ -heterocycloalkyl” refers to saturated or partially unsaturated rings having 3 to 6 atoms and containing at least two N. Examples include pyrazolidinyl-, diazetidine-, hexahydropyridazine-, diazepane- and diazoctane groups. “Pharmaceutically acceptable salts” refers to salts of the compounds of Formula I that retain the desired biological activity. Examples of such salts include, but are not restricted to, acid addition salts formed with inorganic acids (e.g. hydrochloric acid, hydrobromic acid, sulfuric acid, phosphoric acid, nitric acid, and the like), and salts formed with organic acids such as acetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disulfonic acid, and polygalacturonic acid. Said compounds can also be administered as pharmaceutically acceptable quaternary salts known by a person skilled in the art, which specifically include the quaternary ammonium salts of the Formula —NR,R′,R″ ⁺Z⁻, wherein R, R′, R″ is independently hydrogen, alkyl, or benzyl, and Z is a counter ion, including chloride, bromide, iodide, alkoxide, toluenesulfonate, methylsulfonate, sulfonate, phosphate, or carboxylate (such as benzoate, succinate, acetate, glycolate, maleate, malate, fumarate, citrate, tartrate, ascorbate, cinnamate, mandelate, and diphenylacetate).

When employed as pharmaceuticals, the compounds of the invention are typically administered in the form of a pharmaceutical composition. Such compositions can be prepared in a manner well known in the pharmaceutical art and comprise at least one active compound. Generally, the compounds of the invention are administered in a pharmaceutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions of the invention can be administered by a variety of routes including oral, rectal, transdermal, intrathecal, subcutaneous, intravenous, intramuscular, and intranasal. Preferably the compounds of the invention are administered by subcutaneous, intramuscular or intravenous injection or infusion.

In a preferred embodiment of the invention, a compound of the invention is fused to a carrier molecule, a peptide or a protein that promotes the crossing of the blood brain barrier (“BBB”). This serves for proper targeting of the molecule to the site of action in those cases, in which the CNS is involved in the disease. Modalities for drug delivery through the BBB entail disruption of the BBB, either by osmotic means or biochemically by the use of vasoactive substances such as bradykinin. Other strategies to go through the BBB may entail the use of passive diffusion and the use of endogenous transport systems, including carrier-mediated transporters such as glucose and amino acid carriers; receptor-mediated transcytosis for insulin or transferrin; adsorptive-mediated transcytosis. Strategies for drug delivery behind the BBB further include intra-cerebral implantation.

Depending on the intended route of delivery, the compounds may be formulated as injectable or oral compositions. The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include pre-filled, pre-measured ampoules or syringes of the liquid compositions or pills, tablets, capsules or the like in the case of solid compositions. In such compositions, the compound of the invention is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or non-aqueous vehicle with buffers, suspending and dispensing agents, colorants, flavours and the like. Solid forms may include, for example, any of the following ingredients, or compounds of a similar nature: a binder such as microcrystalline cellulose, gum tragacanth or gelatine; an excipient such as starch or lactose; a disintegrating agent such as alginic acid, Primogel, or corn starch; a lubricant such as magnesium stearate; a glidant such as colloidal silicon dioxide; a sweetening agent such as sucrose or saccharin; or a flavouring agent such as peppermint, methyl salicylate, or orange flavouring.

Injectable compositions are typically based upon injectable sterile saline or phosphate-buffered saline or other injectable carriers known in the art. The above-described components for orally administered or injectable compositions are merely representative. Further materials as well as processing techniques and the like are known to the skilled practitioner.²¹
²¹Gennaro, A. R., et al., Remington's Pharmaceutical Sciences, Part 8; 18th ed. Easton: The Mack Publishing Company, 1995

The compounds of this invention can also be administered in sustained release forms or from sustained release drug delivery systems. A description of representative sustained release materials is also known to the skilled practitioner.^{22,23,24}
²²Oral Sustained Release Formulations: Design and Evaluation; by Avraham Yacobi, Eva Halperin-Walega (Editor); 1st Ed. edition; Pergamon Press (April 1988)

²³Sustained-Release Injectable Products; Judy Senior (Editor); Interpharm Press (July 2000)

²⁴Encapsulation and Controlled Release; by D. R. Karsa, R. A. Stephenson (Editor); Springer Verlag (December 1993)

The compounds of the invention prevent the aggregation of Aβ associated with the onset and progression of Alzheimer's disease, Dementia Pugilistica (including head trauma), Hereditary Cerebral Haemorrhage with amyloidosis of the Dutch type (HCHWA-D) and Vascular Dementia with amyloid angiopathy. In a preferred method of use of the compounds, administration of the compounds is by injection or infusion, at periodic intervals. The administration of a compound of the invention should preferably begin before any symptoms are detected in the patient, and should continue thereafter. Patients at a high risk for developing Alzheimer's disease, Hereditary Cerebral Haemorrhage with amyloidosis of the Dutch type (HCHWA-D) and Vascular Dementia with amyloid angiopathy include those with a familial history of these diseases.

Still a further aspect of the present invention is a process for preparing aza-peptides comprising the steps of:
a) Reacting an aza-amino acid building block of Formula (II) with an amino acid, an aza-amino acid, a peptide, an aza-peptide or an azatide to form a aza-peptoidic bond through aza-peptide coupling:

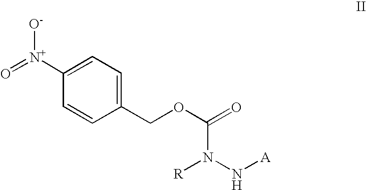

wherein R is selected from H and C₁-C₆ alkyl;

A is any functional group of an amino acid, optionally protected by a protecting group;

R and A can form an optionally substituted C₃-C₆ -heterocycloalkyl ring, preferably pyrazolidinyl-, diazetidine-, hexahydropyridazine-, 1,2-diazepane-, or 1,2-diazoctane group.
b) Removing the para-nitro carbobenzyloxy group.

Typically preferred conditions of aza-peptide coupling for step a) from the process above is the use of carbonyl realeasing agents like triphosgene in the presence of a tertiary base such as DIEA for preactivating the aza-amino acid at low temperature. Typically preferred conditions of removal of the para-nitro carbobenzyloxy group (step b) from the process above are the use of a reducing agents selected from Stannous (II) chloride dihydrate, sodium dithionite and the presence of palladium as catalyst hydrogen gas. Most preferred is the use of SnCl₂x2H₂O.

An another aspect of the invention is a process as described above wherein at least one aza-amino acid building block in step b) is selected from Formulae III, IV, V and VI:

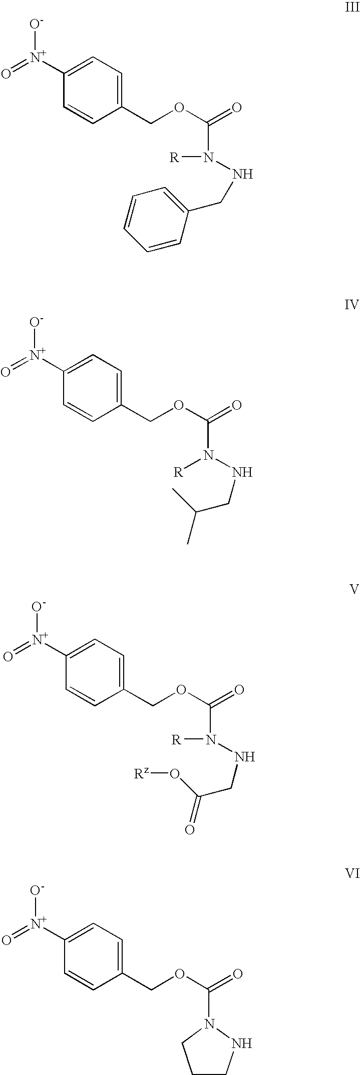

wherein R is selected from H and C₁-C₆ alkyl, preferably H;

R^{Z} is selected from —CH₂—CH═CH₂ and -tert-butyl.

Another preferred embodiment of the invention consists in a process as described above for the preparation of an aza-peptide of a total sequence from 2 to 10 peptoid units in length, preferably from 2 to 5, most preferably 5.

Another preferred embodiment of the invention consists in a process as described above for the preparation of an aza-peptide having between 2 to 10 aza-amino acids, preferably 2 to 5, most preferably 1 or 2.

Another preferred embodiment of the invention consists in a process as described above for the preparation of an aza-peptide of Formula I.

A further aspect of the invention is a synthetic aza-peptide building block having a Formula selected from Formulae III to VI.

It will be appreciated that where typical or preferred experimental conditions for preparing compounds of Formula I (i.e., reaction temperatures, time, moles of reagents, solvents, etc.) are given, other experimental conditions can also be used unless otherwise stated. Optimum reaction conditions may vary with the particular reactants or solvent used, but such conditions can be determined by one skilled in the art by routine optimisation procedures.

The compounds of the invention may be prepared using methods of peptide synthesis known to the skilled practitioner.^{25,26} In a preferred embodiment, the compounds of the invention are synthesised using solid-phase methods.
²⁵Principles of Peptide Synthesis (Springer Laboratory); Miklos Bodanszky, Milkos Bodanszky; 2^{nd} revised edition; Springer Verlag;

²⁶Fmoc Solid Phase Peptide Synthesis: A Practical Approach; Weng C. Chan (Editor), Peter D. White (Editor); Oxford University Press;

A preferred route to the compounds of the invention is depicted in Scheme 3, and particular examples are given in the Examples that follow.

Abbreviations

The following abbreviations are hereinafter used in the accompanying examples:

min (minute), hr (hour), g (gram), mmol (millimole), m.p. (melting point), eq (equivalents), ml (milliliter), μl (microliters), ACN (acetonitrile), All (allyl), BOP (benzotriazol-1-yl-oxcy-tris-(dimethylamino)-phosphonium hexafluorophosphate), Boc (butoxycarbonyl), Cbz (carboxybenzyl), DCM (dichloromethane), DIEA or DIPEA (diisopropyl ethylamine), DMAP (4-dimethylamino-pyridine), DMF (dimethyl formamide), EtOAc (ethyl acetate), Et₂O (diethylether), Fmoc (9-fluorenylmethoxy carbonyl), HATU (0-(7-azabenzotriazol-1-yl)-1,1,3,3-tetramethyluronium hexafluoro phosphate), rt (room temperature), TEA (triethylamine), TFA (trifluoro-acetic acid), THF (tetrahydrofuran), NMP (M-methyl-pyrrolidone), cpm (counts per minute), Ci (Curies).

Synthesis of Compounds of the Invention:

The compounds of the invention can be prepared from readily available starting materials using the following general methods and procedures. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used. Such conditions can be determined by the person skilled in the art, using routine optimisation procedures.

General Protocol:

Generally, the aza-peptide/azatide derivatives according to the general Formula (I) can be synthesized using standard peptide synthesis techniques either in solution or on solid phase, as far as peptide coupling step is concerned. In both approaches typical coupling reagents are used, which are known to the person skilled in the art. Examples of Azapeptide synthesis are given by Gante.²⁷
²⁷Gante, Synthesis, 6(1), 1989, 405-413

It will be also appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions will be appreciated by the person skilled in the art. A general synthesis pathway is represented on scheme 1 below.

According to the position and the number of the aza-amino acid(s) desired in the aza-peptide sequence, the aza-peptides of the invention can be synthesized following the left side of scheme 1 (coupling of aza-amino acid) or following the right side of scheme 1 (coupling of a “normal” amino acid) and this recurrently all along the (aza)peptide sequence.

A generic example is given on scheme 1 for the second (aza)amino acid coupling to the first (aza)amino acid (A) to give a di-(aza)peptide (B).

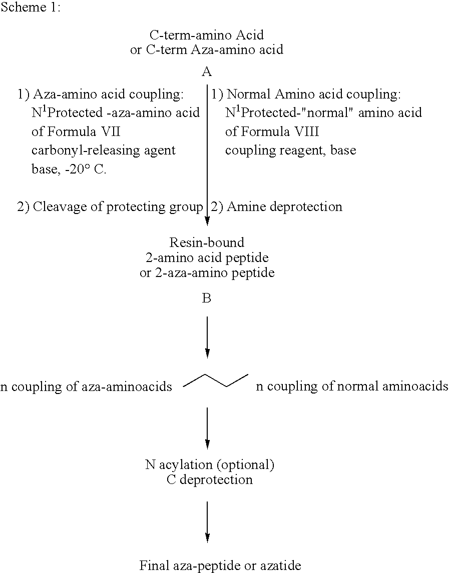

Aza-peptides of Formula I are synthesized by using the left protocol of scheme 1 at least for one coupling step.

Azatides are obtained by following only the left side of scheme 1 for each coupling step.

The coupling step of the aza-amino acid is performed using the corresponding N¹-protected-N²alkylated hydrazine derivative of Formula VII (building block), in the presence of a carbonyl-releasing agent (pre-activator) and a base (scheme 1, step 1, from left protocol). 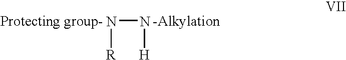
wherein “alkylation” on the hydrazine represents the side-chain of the corresponding amino-acid (e.g. alkylation=benzyl (Bn) is corresponding to the aza-aminoacid, i.e. aza-phenylalanine);

the “protecting group” of the hydrazine is an amino protecting group which can ultimately be cleaved without loss of the structure (Wuts and Greene, 3^{rd} edition). Preferred protecting groups are tert-butyloxy-carbonyl, 4-nitrobenzyloxy-carbonyl (p-NO₂-Z). R is the lateral chain of the nitrogen group.

The coupling step of the normal amino acid is performed using the corresponding normal amino acid of Formula VIII, using classical peptide synthesis (scheme 1, step 1, from right protocol).

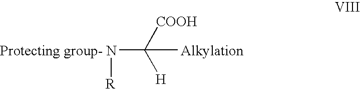

In a preferred embodiment the aza-peptides of Formula (I) may be synthesized on a solid support following the description for scheme 1 above.

A generic example is given on scheme 2 below for the second (aza)amino acid coupling to the first (aza)amino acid bound to the resin (A) to give a di-(aza)peptide (B).

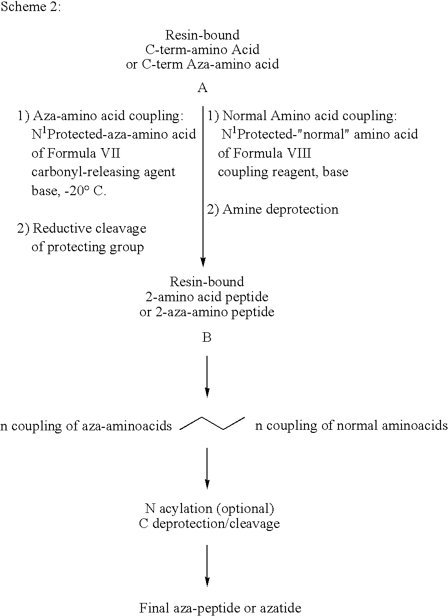

In this case, the “protecting group” of the hydrazine of Formula VII is an amino protecting group which can ultimately be cleaved without loss of the structure or release of the corresponding product from the resin (Wuts and Greene, 3^{rd} edition).

The synthesis can be performed by using, for example, preferred Rink-Amide resin leading to C-terminal amide. Preferred protecting groups are 4-nitrobenzyl-oxycarbonyl (p-NO₂-Z) for the aza-aminoacids, to accomplish the entire synthesis of C-amidated aza-peptides on resin.

The para-nitro carbobenzyloxy group is removed preferably using reducing agents selected from Stannous (II) chloride dihydrate, sodium dithionite and the presence of palladium as catalyst hydrogen gas. Most preferred is the use of SnCl₂x2H₂O.²⁸
²⁸Peluso et al. Journal of Organic Chemistry, 64(19), 1999, 7114-7120

A preferred pathway for preparing penta-aza-peptides according to the general Formula I, wherein R¹, R², R³, R⁴, R⁵, R⁶, R^{7,} R⁸, R⁹, X₁, X₂, X₃, X₄ and X₅ are defined above is described in scheme 3, below:

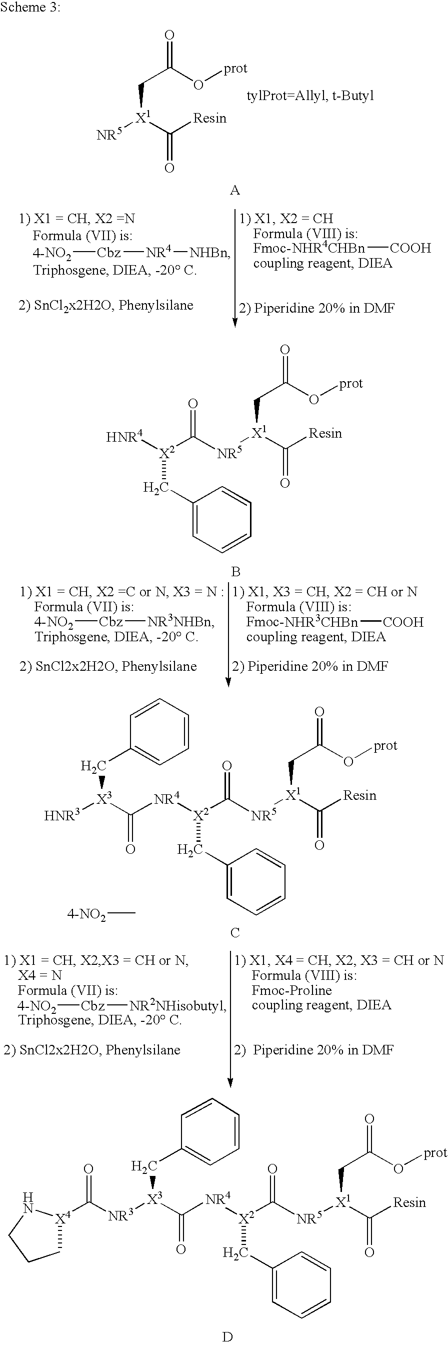

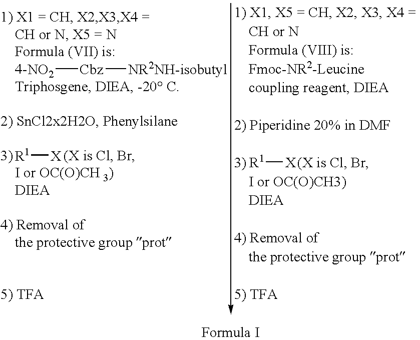

The carbonyl-releasing agent used in the coupling of the aza-aminoacid is defined as a chemical substance that contains a carbonyl-group adjacent to leaving groups, such as Carbonyldiimidazole, bis(4-nitrophenyl) carbonate, bis(2,4-dinitrophenyl)carbonate, (bis(trichloromethyl)carbonate), bis(pentafluorophenyl) carbonate, 4-nitrophenylchioroformate, chloroformic acid, trichloromethyl ester, preferably chloroformic acid-trichloromethyl ester, most preferably bis(trichloromethyl)carbonate (triphosgene). The base used in the coupling of the azaaminoacid can be any type of tertiary amine, capable of scavenging the releasing protic leaving groups, such as N-Me-pyrrolidine, triethylamine, diisopropylethylamine or any tertiary base of similar pKa.

Typically the coupling reaction of the aza-amino acid is conducted in such a way that the hydrazine derivative of Formula VII (1.5 to 2 equivalents referring to the peptide, aza-peptide or azatide) is pre-activated under inert atmosphere during 15 min to 2 hours, with the carbonyl-releasing agent (1 to 1.2 equivalent referring to the hydrazine derivative) in an inert solvent, such as tetrahydrofurane, dioxane, dichloromethane, chloroforme, preferably in tetrahydrofurane, at −50° to −10° C., preferably at −20° C.

This mixture is subsequently added to N-terminal free amino acid, peptide, aza-peptide or azatide, and stirred for 2 to 15 hours, preferably 3 hours.

In case where the amino acid, peptide, aza-peptide or azatide is attached to resin, the resin is washed with 5% of N-Me-pyrrolidine, triethylamine, diisopropylethylamine or any tertiary base of similar pKa in DCM for 5 minutes prior to the addition of the pre-activated hydrazine building block.

The reaction mixture is then carefully washed with different organic solvents, typically used in solid phase organic synthesis.

The N¹-protected-N²alkylated hydrazine derivatives used for the coupling of the aza-amino acid are of Formula (VII), wherein the “alkylation” is —CHR^{s} and wherein —CHR^{s} represents the side chain of the corresponding amino-acid, can also be represented by Formula (VII′).

These N¹-protected-N²alkylated hydrazine derivatives are derived from the corresponding hydrazone derivatives of Formula (IX) using palladium catalyzed hydrogenation procedures, known to the person skilled in the art as shown in scheme 4:

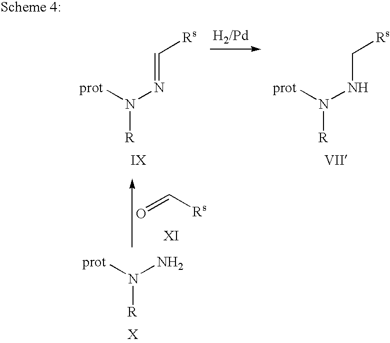

Typically, the corresponding N¹-protected-N²alkyl-hydrazone derivative of Formula (IX) is dissolved in an organic solvent suitable for hydrogenations, followed by the addition of palladium or palladium on charcoal as shown on scheme 4. The mixture is stirred in a hydrogenation apparatus and the apparatus is charged with hydrogen of 2 to 15 bars. The stoechiometric uptake of hydrogen is monitored.

The corresponding hydrazones of Formula (IX) are themselves derived from the corresponding N¹-protected-hydrazines of Formula (X) which are reacted with the alkyl carbonyl derivative of Formula (XI) carrying the desired alkyl moiety (scheme 4). Typically a N¹-protected-hydrazine of Formula (X) is mixed with the corresponding carbonyl derivative of Formula (XI) in an inert solvent such as acetone, tetrahydrofurane or any other solvent used by the person skilled in the art. Typically, the hydrazone precipitates out of the solvent mixture.

In cases where the desired alkyl moiety is incompatible with the ultimate hydrogen step, a direct alkylation of the N¹-protected-hydrazines of Formula (X) may be applied (see scheme 5).

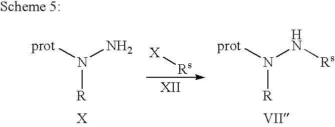

Typically, N¹-protected-hydrazines (X) are reacted with the corresponding alkyl-halides of Formula (XII), wherein X is a halogen, in the presence of a base as scavenger. In order to avoid overalkylation, the N¹-protected-hydrazines are used in excess from 1.2 to 5 equivalents. The resulting N¹-protected-N²alkylated hydrazine derivative of Formula (VII″) or of Formula (VII) wherein the “alkylation” is —R^{s} and —R^{s} represents the side chain of the corresponding amino-acid, is purified using standard chromatographic techniques.

Azaproline derivatives may be synthesized from N¹,N²-bis-protected-hydrazines of Formula (XIII) as illustrated on scheme 6 below:

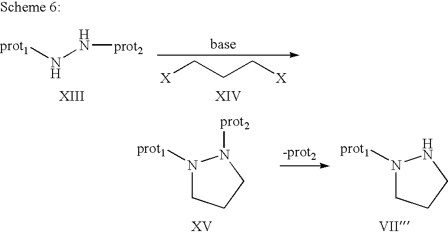

The cyclization step may be achieved using 1,3-bis-halogen-propane derivatives of Formula (XIV) wherein X is an halogen, in the presence of a strong base like KH, K-OtBu, preferred NaH in an inert solvent like THF, dioxane, DMF, DMA or NMP at −20° C. to 20° C.

Typically, a di-anion species is generated in a first step before adding the 1,3-bis-halogen-propane derivative of Formula (XIV) yielding in N¹,N²-bis-protected-azaproline derivative of Formula (XV). Appropriate removal of one protected group gives rise to the corresponding N¹-protected-azaproline derivatives of Formula (VII′″) or of Formula (II) wherein R and the “alkylation” moiety are forming a pyrazolidinyl ring together.

In the case where the amino acid, peptide, aza-peptide or azatide is in solution the reaction mixture is worked up using standard procedures and the crude is purified by flash-chromatography.

When the cycle of N-term deprotection-coupling-deprotection-coupling is performed to lead to the pentapeptide of the invention, a final deprotection step can be followed by an N-term acylation when the desired compound of the invention is blocked at the N-term. This step will be skipped in case of a compound of the invention with a free N-term.

A final deprotection of the C-term of the peptide of the invention is then followed by cleavage from the resin to yield to a compound of the invention with an amidated C-terminus.

Purifications are performed as followed: Preparative HPLC Waters Prep LC 4000 System equipped with columns Prep Nova-Pak®HR C186 μm 60 Å, 40×30 mm (up to 100 mg) or 40×300 mm (up to 1 g). All the purifications were performed with a gradient of MeCN/H₂O 0.09% TFA.

For the synthesis of free C-term aza-peptides, Wang or Sasrin® resin are preferred. The process is then similar to the process described above.

Repeating the coupling of only aza-amino acids at each coupling step will lead to azatides.

Azatides can be synthesized in a preferred manner in solution, using preferably t-butyloxycarbonyl as protection group for the aza-aminoacid. Azatides can also be synthesized in liquid phase following know protocols, an example is illustrated by Han et Janda²⁹.
²⁹Han H. and Janda, K. D., Journal of the American Chemical Society, 118, 1996, 2539-2544

The following building blocks are commercially available from Bachem or Novabiochem, Switzerland: Fmoc-L-Phenylalanine, Fmoc-L-Proline, Fmoc-L-Leucine and Fmoc-D(OAll)-OH.

NO₂-Z-NH—NH-Bn (N¹-(4-Nitrobenzyloxy-carbonyl)-N²-benzyl-hydrazine), NO₂-Z-aza-proline (Pyrazolidine-1-carboxylic acid-(4-nitrobenzyl)ester), NO₂-Z-NHNH-isobutyl (aza-leucine), N¹-(4-Nitrobenzyloxy-carbonyl)-N²-isobutyl-hydrazine, NO₂-Z-NH—NH—CH₂COOH (aza-aspartic acid), [N¹-(4-Nitro-benzyloxycarbonyl)-hydrazino]-acetic acid were prepared from commercially available materials using the method as cited below.

Coupling reagents are commercially available from Novabiochem, Switzerland.

### EXAMPLES

The compounds of Examples 1 to 7 are preferred embodiments of the invention:

### Example 1

### Building Block: N′-(4-Nitrobenzyloxy-carbonyl)-N²-benzyl-hydrazine (1)

Compound of example 1 is compound of Formula (III) wherein R is H and can be synthesized following schemes 4-5:

### 1) N¹-(4-Nitrobenzyloxy-carbonyl)-N²-(tert.butyloxy-carbonyl)-hydrazine (1a)

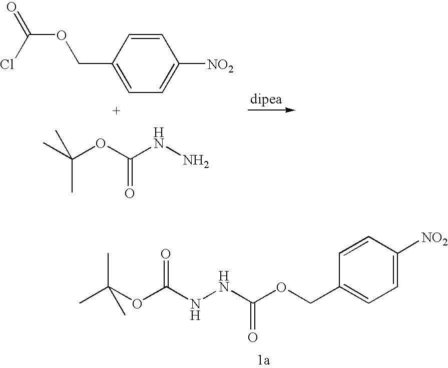

1.23 g (9.28 mmol) of tert-butyl-carbazate and 3.2 ml (15.55 mmol) of dipea were dissolved in 50 ml of DCM. To this solution was added dropwise a solution of 2 g (9.28 mmol) of 4-nitrobenzyl chloroformate in 50 ml of DCM.

The mixture was magnetically stirred for 1 hour. The solution was washed with HCl 0.1 N, the organic phase was dried with magnesium sulfate and concentrated under vacuum affording a yellowish solid which leads to compound 1a (2.45 g, 86%.yield). LC-MS; (M-Boc+1)⁺=212; (M−1)⁻=310.2. ¹H-NMR: (CDCl3, 300 Mz), δ: 1.47 (s, 9H,); 5.32 (s, 2H); 6.45 (bs, 1H,) 6.82 (bs, 1H); 7.02 (d, 2H), 8.23 (d, 2H). ¹³C-NMR: (CDCl3, 300 Mz) δ: 28.50; 66.46; 82.505; 121.16; 128.56; 143.41; 148.10; 154.00; 170.30.

### 2) N¹-(4-Nitrobenzyloxy-carbonyl)-hydrazine (1b)

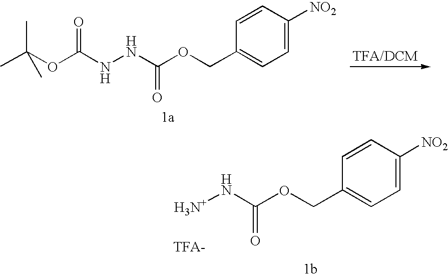

2.4 g of compound la were dissolved in 10 ml of DCM/TFA (25%). The mixture was stirred for two hours, the solvent evaporated and 5 ml of diethyl ether were added, while the desired product crystallizes (compound 1b). The crystals were filtered and washed with ether. LC-MS; (M+)⁺=212; (M−1)⁻=210.26.

### 3) N¹-(4-Nitrobenzyloxy-carbonyl)-N²-benzyl-hydrazine (1)

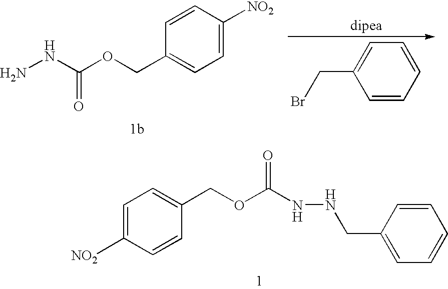

5.2 g of compound 1b and 6.9 ml of dipea were dissolved in 120 ml of ethanol. To this solution was added a solution of 2.4 ml of benzylbromide in 50 ml of ethanol. The mixture was refluxed overnight, after that the solvent evaporated under vacuum and 150 ml of ethyl acetate were added. A white solid was filtered off and the filtrates were concentrated and purified by flash chromatography using cyclohexane: ethylacetate; 8:2 as eluents. 4.4 g (72%) of desired mono-alkylated product (1) could be isolated. ¹H-NMR: (DMSO, 300 Mz), δ: 3.38 (bs, 1H,); 4.13 (s, 2H); 5.1 (s, 2H,) 7.58 (d, 2H); 8.25 (b,7H); 8.48 (bs, 1H).

### Example 2

### Building Block: Pyrazolidine-1-carboxylic acid-(4-nitrobenzyl)ester (2)

Compound of example 2 is compound of Formula (VI) or of Formula (VII′″) wherein Prot1 is 4-NO₂-Cbz and can be synthesized following scheme 6:

### 1) N¹-tert.butyloxy-carbonyl-N²-benzyloxy-carbonyl-hydrazine (2a)

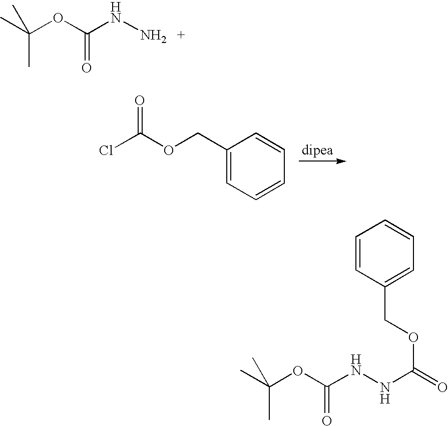

10 g of commercially available tert-butyl-carbazate were dissolved in 400 ml of DCM, to which was added dropwise a solution of 25.9 ml of DIPEA in 100 ml of DCM, followed by the addition of a solution of 11.88 ml of benzyloxycarbonyl-chloride in 100 ml of DCM. This mixture was magnetically stirred overnight. After that the solution was washed with HCl 0.1 N. and with brine. The organic phase was dried with magnesium sulfate and concentrated under vacuum, affording a yellow oil (2a) (20.2 g, 99%). LC-MS; (M−1)⁻=265.22. ¹H-NMR: (DMSO, 300 Mz) δ: 1.48 (s, 9H,); 5.19 (s, 2H); 6.39 (bs, 1H,) 6.63 (bs, 1H); 7.37 (m, 5H). ¹³CNMR: (DMSO, 300 Mz) δ: 28.51; 68.156; 82.24; 128.94; 136.00; 158.40.

### 2) Pyrazolidine-1,2-dicarboxylic acid1-benzyl ester 2-tert-butyl ester (2b)

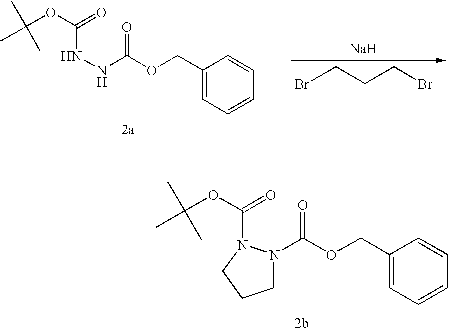

In a well dried, 500 ml three necked flask, 1.5 g of NaH (60%) were suspended in 100 ml of DMF. The mixture was cooled at 0° C. A solution of 3.9 g of tert-butyl carbazate (2a) in 100 ml of DMF was added dropwise. The mixture turned red. A solution of 1.91 ml of 1,3 dibromopropane in 100 ml of DMF was added and the reaction was stirred for 30 minutes at 0° C. and overnight at r.t.

The solvent was evaporated to dryness and the solid was washed with ethyl acetate (100 ml). A white powder was filtered off and the organic layer was washed with brine (3×50 ml) and dried with magnesium sulfate affording a yellow oily residue (5.4 g), which ultimately purified by flash-chromatography. (2b). LC-MS; (M-Boc+1)⁺=207; (M-56−1)⁻265. ¹H-NMR: (CDCl3, 300 Mz) δ: 1.35 (s, 9H,); 1.97 (m, 2H); 3.20 (m, 2H,) 3.85 (m, 2H); 5.10 (m, 2H); 7.27 (m, 5H). ¹³C-NMR: (CDCl3, 300 Mz) δ: 14.58; 28.46; 68.15; 46.66; 60.78; 81.96; 128.45; 136.60; 157.15; 174.10.

### 3) Pyrazolidine-1-carboxylic acid tert.-butyl ester (Boc-Azaproline) (2c)

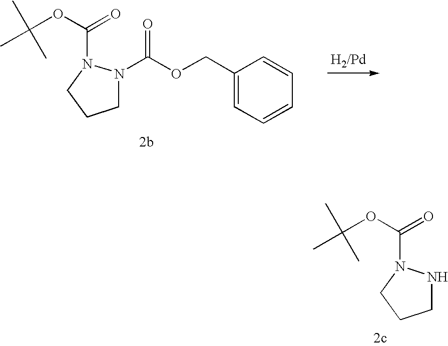

The pyrazolidine-1,2-dicarboxylic-acid1-benzylester-2-tert-butylester (2b) was dissolved in MeOH. A pressure of hydrogen of 110 psi was applied and the reactor was stirred over night. The crude obtained was sufficiently pure for further reactions (yield 60%) (2c). ¹H-NMR: (CDCl3, 300 Mz) δ: 1.42 (s, 9H); 1.95 (m, 2H); 3.00 (m, 2H,) 3.36 (m, 2H); 3.75 (bs, 1H). ¹³C-NMR: (CDCl3, 300 Mz) δ: 28.41; 28,85; 45.96; 48.17; 80.57; 155.45.

### 4) Pyrazolidine-1,2-dicarboxylic acid1-(4-nitrobenzyl)ester 2-tert-butyl ester (2d)

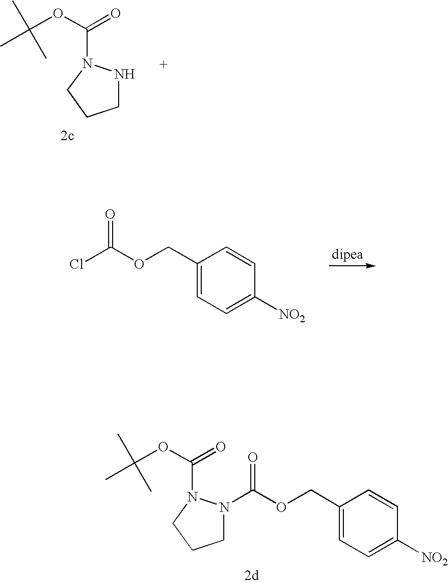

The synthesis of compound (2d) was carried out according to the same protocol used in step 1) for obtaining compound (2a), reacting compound (2c) in presence of 4-nitro benzyloxycarbonyl chloride as shown above. The crude was purified on silicagel using cyclohexane: THF (60:40) as eluents. (yield 43%) leading to compound (2d). LC-MS; (M+1)⁺=352.22. ¹H-NMR: (CDCl3, 300 Mz) δ: 1.39 (s, 9H); 2.00 (m, 2H); 3.25 (m, 2H) 3.88 (m, 2H); 5.20 (m, 2H), 7.47 (d, 2H), 8.14 (d, 2H). ¹³CNMR: (CDCl3, 300 Mz) δ: 26.00; 28.50; 46.53; 47.51; 66.52; 82.27; 124.06; 128.52; 41; 144.10; 147.95; 156.48, 156.59.

### 5) Pyrazolidine-1-carboxylic acid-(4-nitrobenzyl)ester (2)

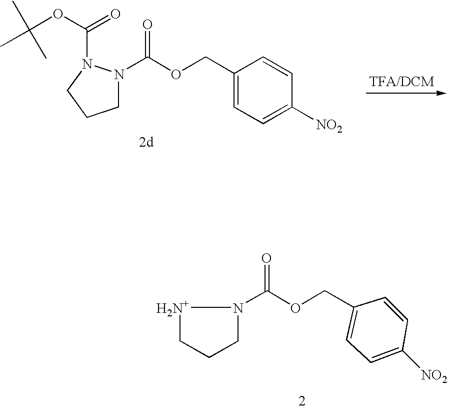

3.2 g of Pyrazolidine-1,2-dicarboxylicacid-1-(4-nitrobenzyl)ester-2-tert-butylester (2d) were dissolved in 15 ml of DCM, to which was added dropwise 5 ml of TFA. The reaction mixture was stirred for 2 h, 150 ml of ethyl ether were added affording a white solid (2). LC-MS; (M+1)⁺=252.33 ¹H-NMR: (CDCl3, 300 Mz) δ: 2.13 (m, 2H); 3.26 (m, 2H,) 3.58 (m, 2H); 5.32 (m, 2H), 7.67 (d, 2H), 8.23 (d, 2H), 9.3 (bs, 1H). ¹³C-NMR (CDCl3, 300 Mz) δ: 25.69; 46.35; 46.78; 66.22; 123.87; 128.67; 144.29; 147.48; 153.76.

### Example 3

### Building Block: N¹-(4-Nitrobenzyloxy-carbonyl)-N²-isobutyl-hydrazine (3)

Compound of example 3 is compound of Formula (IV) wherein R is H and can be synthesized following schemes 4-5 as described above for compound (1).

### Example 4

### Building Block: [[N1-(4-Nitro-benzyloxycarbonyl)-hydrazino]-acetic acid tert-butyl ester (4)

Compound of example 4 is compound of Formula (V) wherein R is H and R^{Z} is -tert-butyl. It can be synthesized following schemes 4-5 as described above for compound (1).

### Example 5

### Ac-L-P-F^{a}-F-D-NH2 (5)

Compound of example 5 is synthesized following scheme 3, respectively following coupling protocol from the right for the first coupling step, then the protocol from the left for the second coupling, then protocols from the right for the remaining last two couplings. The synthetic conditions are as follows:

### 1) Last Amino Acid Attachment to the Resin: Formation of Pol-D(All)-Fmoc (5a)

The last amino acid of the aza-peptide of the invention is first bound to the resin to form a resin-bound amino acid of Formula A in scheme 3 (Formula 5a) as follows:

In a 500 ml solid phase reactor 11.7 g of Rink resin NOVASYN(R) TGR resin.LL (0.23 mmol/g) was shaken for 5 minutes twice with 200 ml of DMF and twice with DCM.

In a test tube some beads of resin beads, three drops of each reagent of the KAISER TEST kit were heated to 120° C. for 5 minutes, the blue product was considered positive.

In a 250 ml solid phase reactor, 11 g of resin were suspended in 100 ml of NMP. 1.8 g (1.5 eq) of Fmoc-D(OAll)-OH, 2.05 g (2.0 eq) of HATU, and 1.74 g (5 eq) of DIPEA, were dissolved in 100 ml of NMP and added to the resin suspension.

The mixture was rocked for 2 h and the resin (Formula 5a) was washed with DMF (3×200 ml) and DCM (3×200) five minutes each washing. (Kaiser test negative).

### 2) Deprotection of the Amine: Formation of Pol-D(All)NH2 (5b)

The above described resin (5a) was shaken in 300 ml Piperidine (25% in DMP) for 2 hours, followed by washing with DMF (3×200 ml, 5 min each) and DCM (3×200 ml, 5 min each). (Kaiser test positive). Resin (5b) is obtained.

### 3) Coupling of the 4^{th} Amino Acid: Formation of Pol-D(All)-F-Fmoc (5c)

A freshly prepared solution of 1.56 g of Fmoc-L-Phenylalanine, 2.05 of HATU, and 1.745 g of DIPEA in 100 ml of NMP was added to a suspension of 11.74 g of (5b), and the mixture was shaken for 1 h. The resin was washed with DMF (3×200 ml) and DCM (3×200) five minutes each washing. (Kaiser test negative). Resin (5c) is obtained (compound C in scheme 3).

### 4) Deprotection of the Amine: Formation of Pol-D(All)-F-NH2 (5d)

The above described resin (5c) was shaken in 300 ml piperidine (25% in DMF) for 2 hours, followed by washing with DMF (3×200 ml, 5 min each) and DCM (3×200 ml, 5 min each). (Kaiser test positive). Resin (5d) is obtained.

### 5) Coupling of Aza-Phenylalanine (F^{a}): Formation of Pol-D(All)F-F^{a}-(NO₂)Z (5e)

To a cooled solution (−20° C.) of 350 mg of Triphosgene in 50 ml dry THF was added a solution of 1 g of NO₂-Z-NH—NH-Bn (compound 1) and 2 ml of DIEA in 25 ml THF. This mixture was stirred under N2-atmosphere for two hours, followed by the addition to a suspension of 7 g of (5d). The reaction solution was slowly warmed to room temperature and stirred for 5 h. Washings: DMF (3×200 ml, 5 min each) and DCM (3×200 ml, 5 min each). (Kaiser test negative). Resin (5e) is obtained. (After cleavage of an aliquot, 5 ml TFA 95%, 2 h), LC-MS, r.t. 1.98 min.; (M+1)+=646.93; (M−1)⁻=644.79.

### 6) Reductive Cleavage: Formation of Pol-D(All)F-F^{a}-NH₂ (5f)

The resin (5e) was swollen in DMF and treated 3 times with 100 ml of SnCl₂-solution (2M SnCl₂; 1.6 mM, AcOH; 0.01 M phenol) in DMF for 2 hours each time to remove the of NO₂-Z protecting group. The resin was then washed sequentially with DMF, DCM: TEA 9:1, DMF, and finally with DCM (2×100 ml, 5 minutes each.; LC-MS; (M+1)⁺=468.27; (M−1)⁻=466.19 (Kaiser test positive) to lead to compound (5f) (Compound C in scheme 3).

### 7) Coupling of the 2^{nd} Amino Acid: Formation of Pol-D(All)F-F^{a}-P-Fmoc (5g):

The resin (5f) was swollen in 200 ml of NMP. A fresh solution of Fmoc-L-Proline (1.5 eq), HATU (2.0 eq) and 1.9 ml (5.0 eq) of DIEA in 200 ml of NMP were added. The reaction was shaken for two hours and washed sequentially with DMF, DCM 3×100 ml 5 minutes each. (Kaiser test negative). Resin (5 g) is obtained.

### 8) Deprotection of the Amine: Formation of Pol-D(All)-F-F^{a}-P-NH₂ (5h)

The above described resin (5g), was shaken in 200 ml piperidine (25% in DMF) for 2 hours, followed by washing with DMF (3×200 ml, 5 min each) and DCM (3×200ml, 5 min each). (Kaiser test positive). Resin (5h) is obtained (compound D in scheme 3).

### 9) Coupling of the 1^{st} Amino Acid: Formation of Pol-D(All)-F-F^{a}-P-L-Fmoc (5i)

The resin (5h) (8.5 g) was swollen in 200 ml of NMP. A fresh solution of Fmoc-L-Leucine (1.21 g, 1.5 eq), HATU (1.74 g, 2.0 eq) and 1.95 ml (5.0 eq) of DIEA in 100 ml of NMP were added. The mixture was shaken for two hours and washed sequentially with DMF, DCM 3×100 ml 5 minutes each. (Kaiser test negative). Resin (5i) is obtained.

### 10) Deprotection of the Amine: Formation of Pol-D(All)-F-F^{a}-P-L-NH₂ (5j)

The above described resin (5i) was shaken in 200 ml piperidine (25% in DMF) for 2 hours, followed by washing with DMF (3×200 ml, 5 min each) and DCM (3×200 ml, 5 min each). (Kaiser test positive). Resin (5j) is obtained.

### 11) Acylation of N-Terminus: Formation of Pol-D(All)-F-F^{a}-P-L-Ac (5k)

8 g of the above described resin (5j) was treated with 1.6 ml of DIEA in 150 ml of DCM To this suspension was added a solution of 2.16 ml of Acetic anhydride in 50 ml DCM. The mixture was stirred for 3 hours and washed with DMF (3×200 ml, 5 min each) and DCM (3×200 ml, 5 min each). (Kaiser test negative). Resin (5k) is obtained.

### 12) Deprotection of the C-Term Amino Acid: Formation of: Pol-D-F-F^{a}-P-L-Ac (5l)

The above described resin (8.5 g, 0.27 mmol/g) (5k) was treated with a solution of 80 ml of DCM and 5.6 ml of PhSiH₃ (4.9 g, 20 eq) for 5 minutes under nitrogen atmosphere. To this suspension was added 530 mg (0.2 eq) of Pd(PPh₃)₄ in 80 ml of DCM. The mixture was shaken for 20 minutes. This cycle was repeated four times, and then the resin was washed with DMF:Water (9:1) 3×150 , DMF 3×150 ml, DCM 3×150 ml for 5 minutes each time. Resin (5l) is obtained.

### 13) Cleavage From the Resin: Formation of: Ac-LP-F^{a}-F-D-NH₂ (5)

The resin (5l) was shaken for 3 h with 150 ml of TFA, then washed with DCM 3×150 ml. The combined filtrate was concentrated under vacuum. The crude was purified by HPLC prep, using acetonitrile/water as eluents, affording compound (5) of Formula I as white powder. LC-MS, r.t. 1.24 min; (M+1)⁺=680.40; (M−1)⁻=678.10.

### Example 6: Ac-L-P^{a}-F-F-D-NH₂ (6)

Compound of example 6 is synthesized following scheme 3, respectively following coupling protocol from the right for the two first coupling steps, then the protocol from the left for the third coupling, then again protocol from the right for the remaining last coupling. The synthetic conditions are as follows:

### 1) Synthesis of Pol-D(All)-F-NH₂ (5d)

Pol-D(All)-F-NH₂ was synthesized according to the protocol for coupling steps used to obtain the intermediate (5d) of compound (5) (Kaiser test positive).

### 2) Coupling of the 3^{rd} Amino Acid: Formation of Pol-D(All)-F-F-Fmoc (6a)

A freshly prepared solution of 1.56 g of Fmoc-L-Phenylalanine, 2.05 of HATU, and 1.745 g of DIPEA in 100 ml of NMP was added to a suspension of 11.74 g of (5d), and the mixture was shaken for 1 h. The resin was washed with DMF (3×200 ml) and DCM (3×200) five minutes each washing. (Kaiser test negative). Resin (6a) is obtained.

### 3) Deprotection of the Amine: Formation of Pol-D(All-F-F-NH2 (6b)

The above described resin (6a) was shaken in 300 ml piperidine (25% in DMF) for 2 hours, followed by washing with DMF (3×200 ml, 5 min each) and DCM (3×200ml, 5 min each). (Kaiser test positive). Resin (6b) is obtained (compound C in scheme 3).

### 4) Coupling of Aza-Proline (P^{a}): Formation of Pol-D(All)-F-F-P^{a}-(NO₂)Z (6c)

To a cooled solution (−20° C.) of 433 mg of triphosgene in 15 ml of THF was added a solution of 1.6 g of pyrazolidine-1-carboxylic acid-4-nitro-benzylester trifluoroacetate ((NO₂)Z-aza-proline×TFA) (compound 2) and 800 microlitres of DIEA in 10 ml of THF. This mixture was stirred under nitrogen atmosphere for two hours, followed by the addition to a suspension of the above described resin (6b) (9 g resin in 1% DIEA/THF, 250 ml). The solution was slowly warmed up to room temperature, and stirred 5 h. Washings: DMF (3×200 ml, 5 min each) and DCM (3×200 ml, 5 min each). (Kaiser test negative). Partial cleavage: LC-MS: r.t. 2.09, (M+1)⁺=745.5; (M−1)⁻=742.81. Resin (6c) is obtained.

### 5) Reductive Cleavage and Amine Deprotection: Formation of Pol-D(All)-F-F-P^{a}-NH₂ (6d)

The resin (6c) was swollen in DMF and treated 3 times with 100 ml of SnCl₂-solution (2M SnCl₂; 1.6 mM, AcOH; 0.01 M phenol) in DMF for 2 hours each time to remove the of NO₂-Z protecting group. The resin was then washed sequentially with DMF, DCM:TEA 9:1, DMF, and finally with DCM (2×100 ml, 5 minutes each; (Kaiser test positive). Resin (6d) is obtained (compound D in scheme 3).

### 6) Coupling of the 1st Amino Acid: Formation of Pol-D(All)-F-F-F^{a}-L-Fmoc (6e)

The resin (6d) (8.5 g) was swollen in 200 ml of NMP. A fresh solution of Fmoc-L-Leucine (1.21 g, 1.5 eq), HATU (1.74 g, 2.0 eq) and 1.95 ml (5.0 eq) of DIEA in 100 ml of NMP were added. The mixture was shaken for two hours and washed sequentially with DMF, DCM 3×100 ml 5 minutes each. (Kaiser test negative). Resin (6e) is obtained.

### 7) Deprotection of the Amine: Formation of Pol-D(All)-F-F-P^{a}-NH₂ (6f)

The above described resin (6e) was shaken in 200 ml piperidine (25% in DMF) for 2 hours, followed by washing with DMF (3×200 ml, 5 min each) and DCM (3×200 ml, 5 min each). (Kaiser test positive). Resin (6f) is obtained.

### 8) Acylation of N-Terminus: Formation of Pol-D(All)-F-F-P^{a}-L-Ac (6g)

8 g of the above described resin (6f) was treated with 1.6 ml of DIEA in 150 ml of DCM To this suspension was added a solution of 2.16 ml of Acetic anhydride in 50 ml DCM. The mixture was stirred for 3 hours and washed with DMF (3×200 ml, 5 min each) and DCM (3×200 ml, 5 min each). (Kaiser test negative). Resin (6g) is obtained.

### 9) Deprotection of the C-Term Amino Acid: Formation of: Pol-D-F-F-P^{a}-L-Ac (6h)

The above described resin (8.5 g, 0.27 mmol/g) (6g) was treated with a solution of 80 ml of DCM and 5.6 ml of PhSiH₃ (4.9 g, 20 eq) for 5 minutes under nitrogen atmosphere. To this suspension was added 530 mg (0.2 eq) of Pd(PPh₃)₄ in 80 ml of DCM. The mixture was shaken for 20 minutes. This cycle was repeated four times, and then the resin was washed with DMF:Water (9:1) 3×150, DMF 3×150 ml, DCM 3×150 ml for 5 minutes each time. Resin (6h) is obtained.

### 10) Cleavage From the Resin: Formation of: Ac-L-P^{a}-F-F-D-NH₂ (6)

The resin (6h) was shaken for 3 h with 150 ml of TFA, then washed with DCM 3×150 ml. The combined filtrate was concentrated under vacuum. The crude was purified by HPLC prep, using acetonitrile/water as eluents, affording compound (6) of Formula I as white powder. (purity 95%). LC-MS: r.t. 1.35, (M+1)⁺=679.99; (M−1)⁻=677.99.

### Example 7

### Ac-L-P^{a}-F¹-F-D-NH₂ (7)

Compound of example 7 is synthesized following scheme 3, respectively following coupling protocol from the right for the two first coupling steps, then the protocol from the left for the third and fourth couplings, then again protocol from the right for the remaining last coupling. The synthetic conditions are as follows:

### 1) Synthesis of Pol-D(All)-F-F^{a}-NH₂ (5f)

Pol-D(All)-F-F^{a}-NH₂ was synthesized according to the protocol for coupling steps used to obtain the intermediate (5f) of compound (5) (Kaiser test positive).

### 2) Coupling of Aza-Proline (P^{a}): Formation of Pol-D(All)F-F^{a}-P^{a}-(NO₂)Z (7a)

Cooled solution (−20° C.) of 433 mg of triphosgene in 15 ml of THF was added a solution of 1.6 g of Pyrazolidine-1-carboxylic acid-4-nitro-benzylester trifluoroacetate ((NO₂)Z-aza-proline×TFA) (compound 2) and 800 microlitres of DIEA in 10 ml of THF. This mixture was stirred under nitrogen atmosphere for two hours, followed by the addition to a suspension of the above described resin (5f) (9 g resin in 1% DIEA/THF, 250 ml). The solution was slowly warmed up to room temperature, and stirred 5 h. Washings: DMF (3×200 ml, 5 min each) and DCM (3×200 ml, 5 min each). (Kaiser test negative). Partial cleavage: LC-MS: r.t. 2.09, (M+1)⁺=745.5; (M−1)⁻=742.81. Resin (7a) is obtained.

### 3) Reductive Cleavage and Amine Deprotection: Formation of Pol-D(All)-F-F^{a}-P^{a}-NH₂ (7b)

The resin (7a) was swollen in DMF and treated 3 times with 100 ml of SnCl₂-solution (2M SnCl₂; 1.6 mM, AcOH; 0.01 M phenol) in DMF for 2 hours each time to remove the of NO₂-Z protecting group. The resin was then washed sequentially with DMF, DCM:TEA 9:1, DMF, and finally with DCM (2×100 ml, 5 minutes each; (Kaiser test positive). Resin (7b) is obtained (compound D in scheme 3).

### 4) Coupling of the 1st Amino Acid: Formation of Pol-D(All)-F-F^{a}-P^{a}-L-Fmoc (7c)

The resin (7b) (8.5 g) was swollen in 200 ml of NMP. A fresh solution of Fmoc-L-Leucine (1.21 g, 1.5 eq), HATU (1.74 g, 2.0 eq) and 1.95 ml (5.0 eq) of DIEA in 100 ml of NMP were added. The mixture was shaken for two hours and washed sequentially with DMF, DCM 3×100 ml 5 minutes each. (Kaiser test negative). Resin (7c) is obtained.

### 5) Deprotection of the Amine: Formation of Pol-D(All)-F-F^{a}-P^{a}-NH₂ (7d)

The above described resin (7c) was shaken in 200 ml piperidine (25% in DMF) for 2 hours, followed by washing with DMF (3×200 ml, 5 min each) and DCM (3×200 ml, 5 min each). (Kaiser test positive). Resin (7d) is obtained.

### 6) Acylation of N-Terminus: Formation of Pol-D(All)-F-F^{a}-P^{a}-L-Ac (7e)

8 g of the above described resin (7d) was treated with 1.6 ml of DIEA in 150 ml of DCM To this suspension was added a solution of 2.16 ml of Acetic anhydride in 50 ml DCM. The mixture was stirred for 3 hours and washed with DMF (3×200 ml, 5 min each) and DCM (3×200 ml, 5 min each). (Kaiser test negative). Resin (7e) is obtained.

### 7) Deprotection of the C-Term Amino Acid: Formation of: Pol-D-F-F^{a}-P^{a}-L-Ac (7f)

The above described resin (8.5 g, 0.27 mmol/g) (7e) was treated with a solution of 80 ml of DCM and 5.6 ml of PhSiH₃ (4.9 g, 20 eq) for 5 minutes under nitrogen atmosphere. To this suspension was added 530 mg (0.2 eq) of Pd(PPh₃)₄ in 80 ml of DCM. The mixture was shaken for 20 minutes. This cycle was repeated four times, and then the resin was washed with DMF:Water (9:1) 3×150 , DMF 3×150 ml, DCM 3×150 ml for 5 minutes each time. Resin (7f) is obtained.

### 8) Cleavage From the Resin: Formation of: Ac-L-P^{a}-F^{a}-F-D-NH₂ (7)

The resin (7f) was shaken for 3 h with 150 ml of TFA, then washed with DCM 3×150 ml. The combined filtrate was concentrated under vacuum. The crude was purified by HPLC prep, using acetonitrile/water as eluents, affording compound (7) of Formula I as white powder. (purity 95%). LC-MS: r.t. 1.35, (M+1)⁺=679.99; (M−1)⁻=677.99.

### Example 8

### Comparative Example 8

The following compound is disclosed in WO 01/34631 (Axonyx Inc.), and is included as a reference compound.

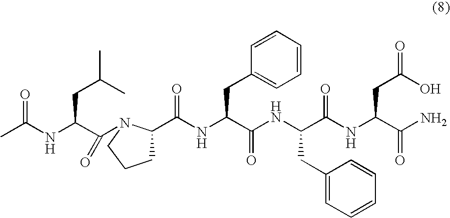

### Example 9

### Biological Assays

In Vitro Assays of Peptide Stability.

The stability of the compounds of the invention can be assayed in comparison with the reference compound (example 8).

Peptides were prepared as a 1 μg/μl solution in water. 20 μl of the peptide solution was diluted in 80 μl of fresh human plasma or 10% rat brain homogenate (prepared in PBS). The resulting solution was incubated at 37° C. for different times and the reaction was stopped by adding a complete cocktail of protease inhibitors. The bulk of plasma and brain proteins (but none of the peptide) were precipitated in cold methanol (mix/MeOH, 4/5, v/v) during one hour at −20° C. The precipitated proteins were pelleted by centrifugation (10000 g, 10 min, 4° C.). The supernatant, containing the peptide, was concentrated 5 times under vacuum and separated by reverse-phase HPLC. The area of the peak corresponding to the intact peptide was measured and compared with an equivalent sample incubated in PBS. The results are listed in Table 1 as percentage of intact peptide after 24 h incubation at 37° C. in human plasma and in rat brain homogenate. The values compare favourably with those obtained for the reference compound of Example 8.

TABLE 1
In vitro stability of various peptides

| | % of intact peptide | % of intact peptide |
|---|---|---|
| Example n° | in human plasma | in rat brain homogenate |
| 5 | 93 | 71 |
| 6 | 93 | 80 |
| 8 | 74 | 2 |

In Vitro Assays of Activity.

The activity of compounds of the invention in inhibiting the formation of aggregated fibrils can be tested by following the changes in fluorescence signal of a fluorophore that has an affinity for the amyloid fibrils.

Amyloid formation can be quantitatively evaluated by the fluorescence emission of thioflavine T (ThT) bound to amyloid fibrils, as reported by Levine³⁰ and also Soto et al.³¹ In this assay, aliquots of Aβ1-42 (a synthetic peptide with the same sequence as the one deposited in the amyloid plaques in Alzheimer's brain) at a concentration of 0.5 mg/ml prepared in 0.1M Tris, pH 7.4 are incubated for 5 days at 37° C., gently swirled on a rotary shaker, in the absence or in the presence of different concentrations of the compounds. At the end of the incubation period, 50 mM glycine, pH 9.2 and 2 μM ThT are added in a final volume of 2 ml. Fluorescence is measured at excitation 435 nm and emission 485 nm in a Perkin Elmer, model LS50B fluorescence spectrometer. Using the analytical method³², the percentage of inhibition of fibril formation caused by compounds of the invention can be calculated.
³⁰Levine, H; Protein science, 2(3), 1993, 404-410

³¹Soto et al, Nature Medicine, 4(7), 1998, 822-6

³²Triguero, D. et al.; J. Neurochem., 6, 1990, 1882-1888.

Cellular Assay of Activity.

Amyloid fibrils are cytotoxic, inducing cell death by apoptosis.¹⁸ The ability of the compounds of the invention in preventing the amyloid formation can be evaluated by measuring the inhibition of the cytotoxicity in a cell assay.
¹⁸U.S. Pat. No. 5,948,763 (New York University)

Aliquots of Aβ1-42 at a concentration of 0.5 mg/ml prepared in 0.1M Tris, pH 7.4 were incubated alone or in the presence of different concentrations of the compounds for 36 h at 37° C., gently swirled on a rotary shaker. At the end of the incubation period, an aliquot of the solution was added to the medium of PC12 cells to reach a final concentration of Aβ of 5.5 μM. The cells were incubated for 24 h and thereafter the cellular viability was evaluated using the MTT kit (Roche, Mannheim, Germany) according to the manufacturer's instructions. Compounds of the invention exhibit an inhibitory activity against amyloid cytotoxicity comparable to that of compound of Example 8.

Blood-Brain Barrier Permeability Studies.

The ability of compound of the invention to cross the BBB can be checked by capillary depletion experiments and the kinetics of their penetration into the brain can be measured.

a) Capillary Depletion and Blood Washout

Capillary depletion can be used to assess the penetration into the brain of compounds of the invention. A “wash-out” step removes blood from the brain so that levels of the compounds of the invention present in the brain parenchyma can be measured.

Capillary depletion studies³² can be done in male CD-1 mice (28-36 g). Mice are anaesthetized with i.p. urethane (40%) and the left jugular vein is exposed. A tritium labelled peptide of the invention is injected i.v.. Before sacrificing the animals, blood is collected from the carotid artery (group 1) or from the descending aorta (group 2). After collection of blood, mice of group 1 are sacrificed or blood is removed by injecting 20 ml lactated Ringer's solution (7.19 g/l NaCl, 0.3 g/l KCL, 0.28 CaCl2, 2.1 g/l NaHCO₃, 0.16 g/l KH₂PO₄, 0.37 g/l MgCl₂.6H₂O, 0.99 g/l D-glucose, 10 g/l bovine serum albumin, pH 7.4) into the left ventricle of the heart for 30 sec, which removes more than 95% of the vascular contents of the brain (blood brain washout, group 2).
³²Triguero, D. et al.; J. Neurochem., 6, 1990, 1882-1888.

The brain/serum ratio (μl/g) is evaluated by the equation: Brain/serum ratio=(cpm/g brain)/(cpm/μl serum). The cerebral cortex is weighed and homogenized in a physiological buffer (10 mM HEPES, 140 mM NaCl, 4 mM KCl, 2.8 mM CaCl₂, 1 mM MgSO₄, 1 mM Na H₂PO₄, and 10 mM D-glucose, pH 7.4). Dextran solution (1.6 ml of a 26% solution) is then added to the homogenate. After centrifugation (5,400 g, 15 min, 4° C.), brain vasculature (pellet) and parenchyma (supernatant) are separated and the radioactivity can be determined in each fraction.

b) Blood Brain Barrier Permeability Study:

The kinetics of penetration of compound of the invention into the brain can be evaluated through blood brain barrier permeability experiments. The percentage of injected peptide found in the brain can then be calculated.

Mice are anaesthetized with i.p. urethane (40%) and the left jugular vein is exposed. 0.2 ml of lactate Ringer's solution (7.19 g/l NaCl, 0.3 g/l KCL, 0.28 CaCl2, 2.1 g/l NaHCO₃, 0.16 g/l KH₂PO₄, 0.37 g/l MgCl₂.6H₂O, 0.99 g/l D-glucose, 10 g/l bovine serum albumin, pH 7.4) containing 1% BSA and tritium labelled peptide (“hot”) is injected. Arterial blood is collected from the right carotid artery at different time points following the labelled peptide injection. Serum is obtained by centrifugation (4800 g, 10 min, 4° C.). Following arterial blood collection, the mice are decapitated and the whole brains, except the pineals and pituitaries, are harvested and weighed. The amounts of radioactivity in brain and serum can be determined after an overnight solubilization step in TS-2 solution (RPI, Mount Prospect, Ill.) at 40° C. The brain serum ratio of total radioactivity can be determined over time after injection. The brain to serum ratio (μl/g) can then be estimated by the equation: Brain/serum ratio=(cpm/g brain)/(cpm/μl serum).

The representation of the brain to serum ratio versus time allows the derivation of the influx rate, Ki (slope) and the volume of distribution (Y intercept), Vi. The influx rate (Ki, microl (serum)/g (tissue weight)-min) represents the rate at which compounds move from the circulation to the brain. The volume of distribution (Vi, microl (serum)/g (tissue weight)) is the apparent volume of material which is distributed to the brain parenchyma at time 0 min.

In Vivo Studies Using an Animal Model of Cerebral Aβ Deposition.

The compounds can be also tested using an in vivo assay, in which inhibition of amyloid deposition in the brain of animals injected with Aβ1-42 by compounds of the invention can be measured.

The inhibitory activity the compound of the invention in the formation of amyloid deposits by can be visualized in vivo by staining cerebral tissue sections with anti-Aβ1-42 antibodies in the presence and absence of a peptide of the invention.

Male Fischer-344 rats weighed 250-300 g and are 3-4 months of age at the time of arrival. The animals are housed 2 per cage, maintained on a 12 h light-dark cycle with access to food and water ad libitum and are habituated to their new environment for 2-3 weeks prior to surgery. Surgery is performed under sodium pentobarbital (50 mg/kg, i.p.) anaesthesia.

Atropine sulphate (0.4 mg/kg) and ampicillin sodium salt (50 mg/kg) are injected sub-cutaneously once the animals are anaesthetized. Aβ1-42 is dissolved in dimethylsulfoxide (DMSO) and then diluted with water to a concentration of 16.7% DMSO. The animal receives a bilateral injection of 5.0 nmol Aβ1-42 into each amygdale by using a Kopf stereotaxic instrument with the incisor bar set at 3.3 mm below the interaural line. Injection coordinates measured from the bregma and the surface of the skull (AP−3.0, ML±4.6, DV−8.8) can be empirically determined based on the atlas of Paxinos and Watson. A volume of 3.0 μl of the solution of Aβ1-42 at 5.0 nmol is administered over 6 min (flow rate 0.5 μl/min) using a CMA/100 micro syringe pump. The cannula is left in situ for 2 min following injection, then it is withdrawn 0.2 mm and left for 3 min, and following these 5 min the cannula is slowly withdrawn. Following surgery the animals is placed on a heating pad until they regained their righting reflex. The animals are then treated with compounds of the invention. The peptides, solubilized in a 0.9% NaCl at the concentration of 4.4 mM are injected s.c (0.5 ml per injection), 4 times a week during 7 and a half weeks.

After treatment with the compounds, animals are sacrificed by an overdose of sodium pentobarbital (150 mg/kg, i.p.), perfused transaortically. For histology, serial coronal sections (40 μm) of the brain are cut, placed in ethylene glycol cryoprotectant and stored at −20° C. until stained. Tissue sections are stained with anti-Aβ1-42 antibodies, as described. An image analysis system is used to determine the size of the amyloid deposits. These data can be analysed by a two-way ANOVA followed by a Newman-Keuls' multiple range test for post hoc comparisons.

1. A compound of the general Formula I: 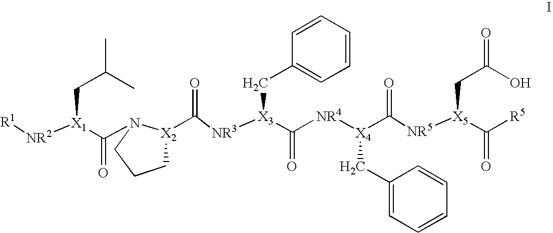
wherein R¹ is selected from H, C₂-C₆ acyl and C₁-C₆ alkyl;
R², R³, R⁴ and R⁵ are independently selected from H and C₁-C₆ alkyl;
R⁶ is selected from OH and NR⁷R⁸, wherein R⁷ and R⁸ are independently selected from H and C₁-C₆ alkyl;
X₁, X₂, X₃, X₄ and X₅ are independently selected from CR⁹ or N wherein R⁹ is selected from H and C₁-C₆ alkyl and with the condition that at least one among X₁, X₂, X₃, X₄ and X₅ is N; as well as any chiral derivative thereof.

2. The compound according to claim 1, wherein R¹ is C₂-C₆ acyl and R⁶ is NR⁷R⁸ wherein R⁷ and R⁸ are independently selected from H and C₁-C₆ alkyl.

3. The compound according to claim 1, wherein X₁, X₂, X₃, X₄ and X₅ are independently selected from CH and N and with the condition that at least one among X₁, X₂, X₃, X₄ and X₅ is N.

4. The compound according to claim 1, wherein R², R³, R⁴ and R⁵ are H and R¹ is —C(O)CH₃ and R⁶ is NH₂; wherein X₁, X₂, X₃, X₄ and X₅ are independently selected from CH and N and with the condition that at least one among X₁, X₂, X₃, X₄ and X₅ is N.

5. The compound according to claim 1, wherein X₂ is N.

6. The compound according to claim 1, wherein X₃ is N.

7. The compound according to claim 1 selected from the following group:
Ac-L-P-F^{a}-F-D-NH2;
Ac-L-P^{a}-F-F-D-NH₂; and
Ac-L-P^{a}-F^{a}-F-D-NH₂.

8. The compound according to claim 1 wherein the compound is utilized as a medicament.

9. A pharmaceutical composition comprising the compound according to claim 1 and a pharmaceutically acceptable excipient, diluent or carrier.

10. A method for the manufacture of a medicament for the treatment or prevention of a disease or condition selected from Alzheimer's disease, Dementia pugilistica (including head trauma), Hereditary Cerebral Haemorrhage with amyloidosis of the Dutch type (HCHWA-D) and vascular dementia with amyloid angiopathy comprising adding the compound according to claim 1 to a medicament.

11. The method according to claim 10, wherein the disease is Alzheimer's disease.

12. A method for the preparation of a medicament for the treatment of a disease associated with abnormal protein folding into amyloid and amyloid-like deposits comprising adding the compound according to claim 1 to a medicament.

13. A process for the preparation of an aza-peptide comprising:
a. reacting at least one an aza-amino acid building block of Formula (II) with an amino acid, an aza-amino acid, a peptide, an aza-peptide or an azatide to form a aza-peptoidic bond through aza-peptide coupling: 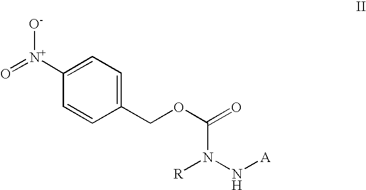
wherein R is selected from H and C₁-C₆alkyl;
A is any functional group of an amino acid;
R and A can form a C₃-C₆ -heterocycloalkyl ring and
b. removing the para-nitro carbobenzyloxy group.

14. The process according to claim 13 wherein the at least one aza-amino acid building block in step a) is selected from Formulae III, IV, V and VI: 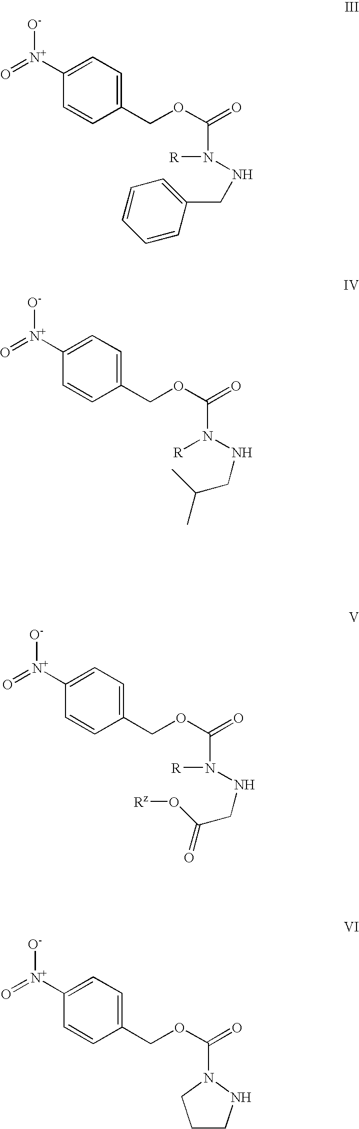
wherein R is selected from H and C₁-C₆ alkyl;
R^{Z} is selected from —CH₂—CH═CH₂ and -tert-butyl.

15. The process according to claim 14 wherein R is H.

16. The process according to claim 13 for the preparation of an aza-peptide of a total sequence from 2 to 10 peptoid units in length.

17. The process according to claim 13 for the preparation of an aza-peptide of a total sequence from 2 to 5 units peptoid units in length.

18. The process according to claim 13 for the preparation of an aza-peptide having between 2 to 10 aza-amino acids.

19. The process according to claim 13 for the preparation of an aza-peptide having between 2 to 5 aza-amino acids.

20. The process according to claim 13 for the preparation of an aza-peptide of Formula I.

21. A synthetic aza-peptide building block having a Formula selected from Formulae III, IV, V and VI.

22. A synthetic aza-peptide building block of claim 21 wherein R is H.

23. A medicament comprising the compound according to claim 1.

24. A method for the treatment or prevention of a disease or condition comprising administering an effective amount of the compound according to claim 1 to a patient in need thereof.

25. A method for the treatment or prevention of a disease or condition comprising administering an effective amount of the compound according to claim 1 to a patient in need thereof, wherein the disease or condition is selected from the group consisting of Alzheimer's disease, Dementia pugilistica (including head trauma), Hereditary Cerebral Haemorrhage with amyloidosis of the Dutch type (HCHWA-D) and vascular dementia with amyloid angiopathy.

26. The method according to claim 25, wherein the disease is Alzheimer's disease.

27. A method for the treatment of a disease associated with abnormal protein folding into amyloid and amyloid-like deposits comprising administering an effective amount of the compound according to claim 1 to a patient in need thereof.


SEQUENCE LISTING (2 sequences)

SEQ ID NO: 1 - protein, 5 aa, Artificial Sequence
  Synthetic Peptide
LPFFD

SEQ ID NO: 2 - protein, 4 aa, Artificial Sequence
  Synthetic Peptide
PFFD